(19) United States
(12) Patent Application Publication
(10) Pub. No.: US 2006/0121474 A1
(43) Pub. Date: Jun. 8, 2006

(54) Bio-chip prepared by gelation on a chip substrate

(75) Inventors: So Youn Kim, Daejeon (KR); Kyun Young Kim, Seoul (KR); Jeong Min Ha, Gyungsangnamdo (KR); Hye Sang Park, Seoul (KR); Young Duk Kim, Daejeon (KR); Phil Seok Kim, Daejeon (KR)

Correspondence Address: BIRCH STEWART KOLASCH & BIRCH, PO BOX 747, FALLS CHURCH, VA, 22040-0747, US

(73) Assignee: LG CHEM, LTD, SEOUL (KR)

(21) Appl. No.: 10/526,402

(22) PCT Filed: Sep. 8, 2003
(86) PCT No.: PCT/KR03/01845

§ 371 Date: Nov. 7, 2005

(30) Foreign Application Priority Data
Sep. 13, 2002 (KR) 10-2002-0055635

(51) Int. Cl. C12Q 1/68; C12M 1/34

(52) U.S. Cl. 435/6; 435/287.2

(57) ABSTRACT

The present invention provides the biochip prepared by the gelation, the preparation thereof, and the method of using the same. The biochip of the present invention is the biochip, unlike the prior biochip with the biomaterials adhered covalently to the surface of the chip substrate, wherein the biomaterials are contained in the pores of the gel-type of spot and encapsulated by the gel-type of spot, said spot being integrated and immobilized on the chip substrate.

### TECHNICAL FIELD

The present invention relates to a biochip prepared by using sol-gel reaction, a method for preparing the biochip and a method for using the biochip.

### BACKGROUND ART

The biochip is a representative example of novel technology combining nanotechnology (NT), biotechnology (BT) and information technology (IT). The biochip is a technology established by combining NT as a material technology, BT as contents and applied field of the material technology and IT as a technology to analyze a large amount of results.

The biochip is formed by high-density micro-arraying of various kinds of biomaterials on a unit area of a surface of a solid supporter and is divided into various types of chips such as a DNA chip, a protein chip, a cell chip, a neuron chip, etc., according to the biomaterials attached onto the surface. Also, the biochip is developed into LOC (Lab-on-a-chip) by combining with the micro-fluids technology.

The biochip includes a technology to immobilize biomaterials, a technology to make biocompatible chip-surface, a technology to micro-array biomaterials, a technology to perform various biological processes on a produced chip, a technology to detect the reaction results, and a technology to modify proteins and genes for production of biomaterials to be immobilized.

The protein chip which the present invention can be applied to is formed by intensive micro-arraying of various proteins on a unit area of a surface of a solid supporter. By using the protein chip, it is possible to conduct with a small amount of samples an experiment of multiple purposes, such as diagnosis of diseases, high throughput screening (HTS), enzyme activity test and the like.

There have been attempts to produce the protein chip by employing the same principles and technical factors for production of DNA chips, which have been already developed and commonly used. Generally, most of the commonly used DNA chips are produced by immobilizing DNA on a glass plate pretreated with a coating material. According to a method similar to that used in the production of DNA chips, when protein is immobilized on a glass plate whose surface is pretreated with a coating material to produce a protein chip, various problems are likely to occur due to differences of physical and chemical properties of the target protein to be immobilized.

Early protein chips were produced by immobilizing proteins onto a surface-treated glass plate and subjected to a simple binding assay. The performance of a protein chip was determined by the activity of the immobilized protein and it was hard to work successfully (See MacBeath and Schreiber [Science 289:1760, 2000]). Such problems are caused by denaturation, inactivation and degradation of proteins resulted from differences of inherent physical and chemical properties of proteins as described above. In order to solve these problems, research and studies have been conducted on the technology of the surface treatment to protein's nature and on materials for immobilizing protein, as are distinguished from those of DNA.

Such research and studies are focused on a method for performing immobilization on a surface of a protein chip while maintaining activity of the protein, including, for example, Hydrogel™ coated slide from Packard Bioscience which has been recently taken over by PerkinElmer, Versalinx chip from Prolinx, PDC chip, a biochip from Zyomyx, etc.

In particular, the hydrogel coated slide is a technology using a 3-dimensional polyacrylamide gel, in which a Swiss glass with an optically leveling silane treated surface is used as a basic supporter material and a surface-modified acrylamide polymer is applied thereon to improve binding force and structural stability of a protein. Here, the protein is immobilized by a covalent bond with a functional group of polyacrylamide gel.

Also, the Versalinx chip of Prolinx comprises a self-assembly monolayer of biotin-conjugated poly(L-lysine)-g-poly(ethylene glycol) formed on a TiO₃ surface, in which a protein is immobilized on the self-assembly monolayered surface, whereby the activity of the protein can be improved.

These methods form a 3-dimensional micro-structure and covalently immobilize proteins on a modified surface so as to maintain activities of proteins within spots. In addition, use of other methods make micro-well type of chips using the microprocessing to produce chips in the solution state.

Meanwhile, the sol-gel process used in the present invention is a technology which has been used to make a micro-structure by the microprocessing, and in particular, has been used in methods comprised of forming a binding net by a mild process and immobilizing biomolecules by another method, not a covalent bond, instead of chemically attaching biomolecules on an inorganic material (See Gill I. and Ballesteros A, [Trends Biotechnol. 18:282, 2000]). Biomolecules including enzymes are immobilized in a mass sol-gel matrix for use in production of a biocatalyst or a biosensor (See Reetz et al. [Adv. Mater. 9:943, 1997]). Specially, it is used in detection of optical color development due to its transparent optical property (See Edminston et al. [J. Coll. Interf. Sci. 163:395, 1994]). Also, biomolecules are known to be not only chemically but also thermally stabilized when they are immobilized on a sol-gel matrix (See Dave et al. [Anal. Chem. 66:1120, 1994]).

In case of the biosensor, the sol-gel reaction is used as a method for patterning by forming a micro structure on a solid supporter as well as for simple immobilization. Here, the patterning method includes shaping the sol in the liquid state using a mold by fluid mechanics, followed by gelation, and separating the mold to form a pattern. For example, a technology designated as micro-moduling in-capillaries (MIMIC) technology is for patterning mesoscopic silica (See Kim et al. [J. Ferment. Bioeng. 82:239, 1995]; Marzolin et al. [Adv. Mater. 10:571, 1998]; Schuller et al. [Appl. Optics 38:5799, 1999]). This technology can be used in basic patterning of micro-fluid engineering.

However, since the activity of protein can be affected by various factors such as pH, it is important to set conditions for the maintenance of the activity when adding the protein from its sol state in the sol-gel process. Thus, technologies for patterning a protein by previously mixing the protein with a sol using various mild conditions such as neutral pH (See Kim et al. [Biotechnol. Bioeng. 73:331 to 337, 2001]) are proposed, but there are problems that the sol-gel process is rapidly progressed to the gel at neutral pH and cracks may occur or the gel turns opaque, according to additives.

### DISCLOSURE OF THE INVENTION

It is an object of the present invention to provide a biochip prepared by using sol-gel reaction, a preparation method of the biochip, and a method for using the biochip.

Until now, there is no technology which can adhere a sol-gel matrix containing biomaterials such as protein in the shape of spots on a chip substrate and thus, there is no biochip comprising the sol-gel matrix integrated in a spot form. By developing a chip substrate surface treatment technology, the present invention can firstly provide a biochip produced using the sol-gel reaction on a chip substrate. By the chip substrate surface treatment technology according to the present invention, a sol mixture containing a biomaterial can be integrated in a spot form on a chip substrate, the sol-gel reaction to gel the sol mixture can occur on a chip substrate, and a sol-gel matrix can be immobilized on a chip substrate.

The present invention provides a biochip wherein a gel type of spots are integrated and immobilized on a chip substrate with biomaterials entrapped in pores of the spot and encapsulated by spot, unlike the conventional biochips in which biomaterials are covalently immobilized on the surface of a chip substrate.

The present invention provides a method for producing a biochip comprising (1) integrating a sol mixture containing biomaterials in the sol state in the shape of spots on a surface treated chip substrate; and (2) gelling the sol mixture in the shape of spots on the chip substrate.

During the gelation of the sol mixture, a 3-dimensional net structure is formed and as a result pores are created. In the pores, the biomaterials are entrapped. Consequently, there can be production of a biochip comprising biomaterials encapsulated in spots in the gel state integrated on a chip substrate.

Also, the present invention provides an assay method of binding between a biomaterial immobilized on a biochip and a target material comprising (1) applying a sample containing the target material to be assayed whether it binds to the biomaterial of the biochip having the biomaterial immobilized by the sol-gel reaction on a chip substrate; and (2) detecting the target material specifically bound to the biomaterial.

The biochip according to the present invention is a new concept of biochip wherein each of spot integrated on a chip substrate forms a carrier having a biomaterial encapsulated in pore therein so that the biomaterial has a free orientation without a covalent bond (See FIG. 8).

Also, the method for producing a biochip by performing the sol-gel reaction with the silicate on a chip substrate for immobilization according to the present invention is a new concept of method for producing a biochip.

Since the biochip according to the present invention is formed by the gelation of a sol mixture containing a biomaterial on a chip substrate, the biomaterial is not covalently bound to a gel matrix, but carried in pores formed in the gel matrix and encapsulated in spots formed of the gel matrix, and thus the biochip improves reactivity.

Therefore, in case that the present invention is applied to a protein chip, a large amount of protein can be contained in spots while maintaining its 3-dimesional structure, whereby it is possible to produce a chip with improved sensitivity. Also, since many proteins can be stabilized by biocompatible additive(s) in a silicate structure which is a basic component of the sol-gel reaction, their activities can be remarkably improved.

(1) Surface Treatment of Chip Substrate

The present invention provides a coating solution for a chip substrate comprising coating agent(s) selected from the group consisting of polyvinyl acetate (PVAc) having a molecular weight in the range of 800 to 200,000, poly(vinyl butyral-co-vinylalcohol -co-vinyl acetate) having a molecular weight in the range of 70,000 to 120,000, poly(methyl methacrylate-co-methacrylic acid) having a molecular weight of 10,000 or more, poly(methyl vinyl ether-alt-maleic anhydride) having a molecular weight of 200,000 or more, poly(methyl vinyl ether-alt-maleic anhydride) having a molecular weight of 1,000,000 or more, poly(methyl acrylate) having a molecular weight of 10,000 or more, 3-glycidoxypropyltrimethoxysilane (GPTMOS), dissolved in solvent(s) selected from the group consisting of methylene chloide (MC), tetrahydrofuran (THF), ethanol, methanol, butanol, methyl ethyl ketone, acetone, isopropyl alcohol (IA), ethyl acetate (EA), methyl isobutyl ketone (MIBK), di-acetone alcohol (DAA) and the like.

The solvent is an organic solvent having a low boiling point.

The solvent is used in a concentration of preferably 5 to 20% by weight of the total coating solution, particularly 5% by weight, 10% by weight, 15% by weight, 20% by weight.

When the above-described coating solution is coated on the chip substrate, the gelation on the chip substrate is promoted, the gel state is not separated in assay on an aqueous phase including antigen-antibody reaction and in severe washing after the gelation, the coating which is of hydrophobic nature can maintain the shape of spots, and since the coating has a high hardness and is optically transparent, it can reduce the background level after reaction. The molecular weight and concentration of the said coating agents was shown to be the most suitable to maintain the above-described properties and performances from experiments.

Also, the present invention provides a substrate for a biochip wherein a chip substrate is coated with the said coating solution. The coating method is preferably spin coating.

Furthermore, the chip substrate useful in the present invention includes the commonly used glass, quartz, silicone, plastic, polypropylene, polycarbonate or activated acrylamide. However, for a measurement and assay by an optical method, the chip substrate is preferred to be optically transparent. Therefore, suitable examples of the chip substrate include optically superior polymers such as poly methyl methacrylic acid (PMMA), polycarbonate (PC), cyclic olefin copolymer (COC) and the like.

The chip substrate can be prepared in a form to react a large amount of sample with many markers.

(2) Preparation of Sol Typed Mixture for Gelation on Chip Substrate

For the present invention, in order to prepare high-density integrated and immobilized spots, having biomaterials such as a protein encapsulated therein, on the surface of the chip substrate via gelation on the chip substrate, a silicate monomer and/or following additives can be used as basic components for the sol-gel matrix.

The additive includes polyglycerylsilicate (PGS), 3-glycidoxypropyltrimethoxysilane (GPTMOS, 98%), (N-triethoxysilylpropyl)-O-polyethylene oxide urethane (PEOU), glycerol, polyethylene glycol (PEG) having a molecular weight in the range of 400 to 10,000 and the like.

The silicate monomer includes tetramethyl orthosilicate (TMOS), tetraethyl orthosilicate (TEOS), methyltrimethoxysillane (MTMS), ethyltriethoxysilane (ETEOS), trimethoxysilane (TMS), 3-aminopropyltrimethoxysilicate (APTMOS) and the like.

In particular, silicate monomers and, PGS, GPTMOS and PEOU among the above-described additives, can perform the sol-gel reaction to form the sol-gel matrix, even when used alone.

Preferably, a mixture of at least one of the silicate monomers and at least one of the additives can be used as a basic component for the sol-gel matrix.

As the basic component for the sol-gel matrix, the mixture of the silicate monomer and/or the additive is used in the range of 30 to 60% by volume of the total sol solution.

The silicate monomer is preferably used in the range of 10 to 40% by volume, more preferably 20 to 40% by volume of the total sol mixture. The additive is preferably used in the range of 2 to 10% by volume of the total sol mixture. If the used amount of the additive exceeds 10% by volume, the compatibility of the sol mixture is deteriorated and the formation of spots on the chip substrate is not well accomplished.

Meanwhile, as shown in Table 1 and 2, considering size of a desired biomaterial, protein activity, sol-gel reaction rate, and morphology of spots, the foregoing additives can be selectively used to correspond to a purpose.

With respect to amounts of the additives in total sol solution, PGS is in the range of 0.5 to 6% by volume, GPTMOS is in the range of 1 to 10% by volume, PEOU is in the range of 5 to 15% by volume, glycerol is in the range of 1 to 5% by volume, and PEG is in the range of 1 to 6% by volume, respectively.

The polyglyceryl silicate (PGS) is a polymerization intermediate from the reaction of silicate monomer and glycerol.

The polymerization intermediate (PGS) plays a critical role in controlling the pore size. The immobilized gel should have an optimal pore size so that the biomaterials (ex., protein) integrated on the biochip surface can readily react with a reactive material. Therefore, the PGS can be preferably added in an amount of 0.5 to 6% by volume of the total sol solution to control the pore size.

The polyglyceryl silicate (PGS) can be prepared by reacting at least one silicate derivative selected from tetramethyl orthosilicate (TMOS), tetraethyl orthosilicate (TEOS), methyltrimethoxysilane (MTMS), ethyltriethoxysilane (ETEOS), trimethoxysilane (TMS), 3-aminopropyltrimethoxy silicate (APTMOS) and the like, as a monomer, with glycerol. The polyglyceryl silicate (PGS) can be prepared according to a method known to the art.

The sol mixture to be gelled on the chip substrate comprises at least one selected from the group consisting of the silicates and the above-described additives and biomaterials (ex. protein) to be integrated on the surface of the chip.

The biomaterials which can be immobilized on the biochip according to the present invention include any biomaterial that can specifically bind to a target material so as to assay the binding therebetween. Preferably, the examples include nucleic acids such as DNA, RNA or PNA, proteins or oligopeptides.

Non-limitative examples of the proteins among the biomaterials which can be high-density integrated on the chip substrate surface according to the present invention include HIV p24, Combo, RgpIII, IgG-Cy3, antigens or antibodies for infectious disease diagnosis, or antigens or antibodies for cancer diagnosis including AFP (Alpha fepto Protein), and enzymes used in activity test. Also, in addition to the proteins, antigens and antibodies, low molecular materials which are used in new drug development can be integrated.

Preferably, the sol mixture may further comprise a pH buffer. As the pH buffer, phosphate buffer can be preferably used and pH can be selected from the range of 4 to 9. Non-limitative examples include pH 5, 5.5, 6, 6.5, 7, 7.5, 8 and 8.5.

The concentration of the pH buffer is preferably in the range of 5 to 100 mM and non-limitative examples include 5, 10, 20, 30, 40, 50, 60, 70, 80, 90 and 100 mM.

For the protein chip, one of the most critical factors to determine the success of the sol-gel process is the time taken for the sol to be the gel and extended for integration. Also, it is critical in production of the protein chip to maintain a suitable viscosity during the sol-gel process by using a proper combination of a composition, thereby producing an optically useful material after gelation.

For the present invention, by controlling the types and composition of the additives added to the sol-gel compound, conditions for gelation (temperature and humidity) and the like, it is possible to delay the time for gelation at maximum 24 hours, based on the conditions specified in the examples of the present invention.

(3) Immobilization of Target Protein on Surface of Chip by Sol-Gel Encapsulation

The present invention provides a biochip produced by applying the sol mixture prepared as described above in spots on a chip substrate, and gelling the spots on the chip substrate to form the biochip wherein biomaterials are entrapped in pores formed by a 3-dimensional net structure of the gel.

The biomaterials are encapsulated in a gel type of spots on the chip substrate and the gel type of spots are immobilized on the chip substrate.

The sol mixture can be integrated on the surface of the chip substrate coated according to the present invention by using a high-density microarraying machine. Here, the conditions for the gelation are a temperature of 4° C. to 25° C. and a humidity of 40 to 80%.

For a protein chip, it is preferable that the spot has a diameter of about 100 to 500 μm and the number of the integrated spots is 1 to 1000 per cm².

In case of a high-density integration, it is possible up to 1,000 spots/cm², though the chip of 100 spots/cm² was prepared in the following example.

The biochip according to the present invention can be applied to new drug screening chips and environmental and toxicity analysis chips as well as protein chips and DNA chips.

(4) Application of Inventive Biochip

The present invention provides a method for assaying a binding between a biomaterial immobilized on a biochip and a target material comprising the steps of applying a sample containing the target material to be assayed for binding to the biomaterial, to the biochip having the biomaterial immobilized by the sol-gel reaction; and detecting the target material specifically bound to the biomaterial.

According to the present invention, the reaction between the biomaterial and the target material occurs in the pores in the gel type of spots wherein the biomaterial is entrapped in the pores and is encapsulated by spot.

For easiness of the detection, the target material can be preferably labeled with a signal inducing material such as a fluorescent dye. The detection of the binding between the biomaterial and the target material can be performed by various methods which are widely used at present, such as a fluorescence detection, an electrochemical detection, a detection using the mass change, a detection using the charge change or a detection using the difference of optical properties, according to the kinds of the signal inducing material attached to the target material.

The biochip prepared by the sol-gel reaction according to the present invention can perform the reactions needed for diagnosis including an antigen-antibody reaction and provide a result of the analysis within 30 minutes to 2 hours, as compared to the conventional immunoassay or biochips.

The biochip according to the present invention can be applied to diseases diagnoses, environmental and toxicity analyses as well as in the basic technologies of the new drug development and acts as a very rapid and sensitive biochip.

The protein chip prepared according to the present invention can be used in a diagnosis, in which an antigen is labeled with a fluorescent dye in the same manner as that used in the Sandwich assay, which is an immunoassay. Here, a fluorescent scanner can be used in the step to measure the result and the diagnosis result can be analyzed and quantified using a program.

The protein chip prepared according to the present invention can be used in the HIV diagnosis.

According to the present invention, since the biomaterial can be added in a mixed sol solution state, proteins or low molecular materials can be highly integrated, whereby it is possible to conduct high throughput screening (HTS) by using the prepared biochip.

Also, since an enzymatic material used in the protein activity test can be integrated in the mixed sol solution, the prepared biochip can be used in the enzyme activity test method. The enzyme for the activity test includes those used in toxicity assay, environment assay and food bacteria assay.

The antigen-antibody diagnosis can be performed automatically in the automatic A-Hyb chamber produced by Memorec or manually in the outside.

(5) Prototypes of Various Products using the Present Invention

By using the gelation on the chip according to the present invention, various kinds of proteins, antigens, antibodies, low molecular materials, and bacteria can be integrated in spots of 10,000 or more at maximum on the chip. As shown in FIG. 7, the present invention can be used representatively in the Blood Bank Screening to screen the transfusion compatibility upon blood collection (infectious disease markers, ex., HIV, I, II, HCV, HBV, Malaria, (H. pylori, Syphillis) (FIG. 7a), and can diagnose a marker for diagnosis of general cancers and concurrently a marker for diagnosis of a specific cancer (FIG. 7b).

### BRIEF DESCRIPTION OF THE DRAWINGS

FIG. 1 shows the result of the sensitivity test of the biochip prepared according to Example 4;

FIG. 2a is a photograph showing the transparency of a spot in the biochip disclosed in Nicholas Rupcich et al. Chem. Mater., 15 (9), 1803 -1811, 2003, and FIG. 2b is a photograph showing the transparency of a spot in the biochip prepared according to Example 4;

FIG. 3 shows the result of the shelf life test of the biochip prepared according to the Example 4;

FIG. 4 is a photograph showing the cross-section of the spot of the biochip prepared according to Example 4 using the Confocal Laser Scanning Microscope (CLSM);

FIG. 5 shows an embodiment, in which an HIV-related indicating protein is integrated by the method for preparing a biochip according to the present invention and the produced chip is applied to a diagnosis;

FIG. 6 shows an embodiment of an AIDS diagnosis using various indicating antigens (p24, combo, rgpIII) and antibodies(anti-p24) for diagnosis of HIV;

FIG. 7 shows prototypes of products prepared by using the present invention. FIG. 7a is two prototypes of diagnosis chip used in the blood examination and FIG. 7b is two prototypes of diagnosis chip used in the cancer diagnosis;

FIG. 8 is a partial schematic view of spots in the biochip according to the present invention.

### BEST MODE FOR CARRYING OUT THE INVENTION

The present invention will be explained in detail by using the following examples. However, the following examples are for illustrative purposes only and the present invention is not limited thereto.

### EXAMPLE 1

### Synthesis of PGS

Tetramethyl orthosilicate (TMOS, 0.048 mole) and methanol (10%) were thoroughly mixed and HCl (0.25 M) was added thereto. The resulting mixture solution was reacted at 70° C. for 6 hours under reflux.

The temperature of the reaction mixture solution was lowered to 50° C. and glycerol (0.192 mole) was added thereto. The resulting mixture solution was reacted at 50° C. for 16 hours. By removal of methanol, polyglycerylsilicate (PGS) was obtained, which was then used for the next process.

### EXAMPLE 2

### Preparation of Protein Chip by Gelation on Chip

A sol containing 20% (g/ml) aqueous solution of polyglycerylsilicate (PGS) synthesized in Example 1 and other additives was applied in spots on a chip substrate and was gelled on the chip substrate to produce a protein chip.

Step 1: Surface Treatment of Chip Substrate

A coating solution of 3% poly(methyl acrylate)/THF was coated on a PMMA slide (76 mm×26 mm) by spin coating. The spin coating was conducted at 500 rpm for 10 seconds and at 1,000 rpm for 40 seconds using Laurell spin coater.

Step 2: Preparation of Sol Mixture

In order to prepare a sol mixture, one addictive selected from polyglyceryl silicate (PGS) aqueous solution, PEOU, PEG, glycerol, GPTMOS and MTMS; tetramethyl orthosilicate (TMOS); and methyltrimethoxysilane (MTMS) were mixed. HCl (final concentration: 5 mM) was added thereto and mixed. Sodium phosphate (final concentration: 10 mM, pH 7), proteins (final amount: 50 pg) and PBS solution (15%) were added and sufficiently mixed. The used proteins are described in detail in Examples 3 to 5.

Step 3: Construction of Protein Chip

The sol mixture prepared in the step 2 was integrated into circular spots having a diameter of 100 to 500 μm on the slide with surface treated in the step 1, by using an inkjet integration program of the Arrayer (Cartesian ), and was left at 25° C. and 80% humidity, as it was, for gelation to produce a protein chip.

### EXAMPLE 3

### Relation Between Composition of Sol Mixture and Biomaterial

The purpose of this example was to seek a composition for optimal performance according to types and size of proteins to be immobilized on the protein chip (for example, according to the size of p24 or BSA protein) or according to use of an antigen or antibody, by using various additives as well as the silicate monomer.

The components and composition of the sol mixture showing the highest sensitivity was determined by criteria that upon reaction with blood, the background level is minimum and the signal is maximum, the gelled proteins are securely attached on the chip during an assay reaction, and the spots have shapes suitable for quantitative analysis. In addition, the criteria includes that the deviation between data should be small for easiness of quality control.

As a result, it was noted that when a small-sized antigen such as P24 was used, the composition 5 was the most suitable under the above-described criteria(see Table 2 below). In particular, using PEG8000 as an additive contributed to formation of spots with the most excellent three dimensional structure. It was possible to set many spots per unit surface area of the slide and after incubation, uniform signal intensity of the encapsulated protein was observed.

When a relatively large size of antigen was used, the composition 8 showed the most excellent performance considering the above-described criteria, unlike the small-size antigen. In particular, it showed uniform appearance in both spot morphology and signal intensity.

TABLE 1
Composition

| | |
|---|---|
| Composition | Composition of Sol mixture |

| No. | TMOS | MTMS | Additive 2-10% |
|---|---|---|---|
| 1 | 25.5% | 12.5% | NO additive |
| 2 | 17.5% | 17.5% | PGS 4% |
| 3 | 20.0% | 10.0% | PEOU 5% |
| 4 | 25.5% | 12.5% | PEG400 3% |
| 5 | 25.5% | 12.5% | PEG8000 5% |
| 6 | 25.5% | 12.5% | Glycerol 2.5% |
| 7 | 25.0% | 7.5% | GPTMOS 5% |
| 8 | 0% | 10% | MTMS |

TABLE 2
Optimal composition of antigen or antibody protein chip

| | Composition of sol | | | | | | |
|---|---|---|---|---|---|---|---|
| | mixture | | Added protein or antibody | | | | |
| Comp. | Silicate | | No | | Anti- | | |
| No. | monomer | Additive | Protein | P24 | P24 | BSA | Result |
| 2 | TMOS +MTMS | PGS | 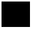 | 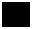 | 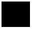 | 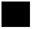 | |
| 3 | TMOS +MTMS | PEOU | 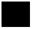 | 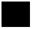 | 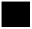 | | |
| 4 | TMOS +MTMS | PEG400 | 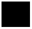 | 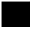 | 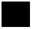 | | |
| 5 | TMOS +MTMS | PEG8000 | 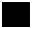 | 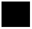 | 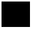 | | X |
| 6 | TMOS +MTMS | Glycerol | 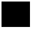 | 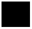 | 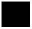 | | |
| 7 | TMOS +MTMS | GPTMOS | 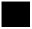 | 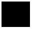 | | | |
| 8 | MTMS | | 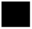 | | 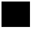 | 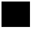 | Y |

X: Optimal antigen chip
Y: Optimal antibody chip

From the result of Table 2, it was noted that the optimal composition for antigen is different from the optimal composition for antibody.

### EXAMPLE 4

### Analysis of Performance of Protein Chip

The protein chip with HIV P24 protein immobilized, prepared by the same method as described in Example 2 using components of the composition 5 in Table 1 was examined for performances including physical properties of the integrated spots, activated states and sensitivities of proteins immobilized in the spots.

Experiment 1: Observation of Cross Section of Spot using CLSM

In order to know whether proteins are present in the gel of the spots and can be 3-dimensionally highly integrated, the spots were tomographically examined by CLSM (Confocal Laser Scanning Microscope). As a result, it was confirmed that the HIV P24 protein was present in the gel and a large amount of the protein was integrated, as shown in FIG. 4.

Experiment 2: Measurement of Maximum Sensitivity of Protein Chip

In order to perform an antigen-antibody reaction using serum from the practical blood treatment, whether the encapsulated protein can remain intact on the chip surface under various reaction conditions, and whether the signal is expressed not randomly but precisely only by the antigen-antibody reaction were examined. Cy3-labeled Cy3-conjugated anti-rabbit IgG (Sigma-Aldrich Company) was added to the sol mixture (all the compositions described in Example 2), instead of the protein and was gelled on the chip substrate to prepare a chip. The produced chip was subjected to a primary antibody reaction, washing, secondary antibody reaction, washing and drying, following the same procedures with the conventional diagnosis method. Upon observation on a scanner, it was confirmed that the signal was clear and quantitatively several thousands times higher, as compared to the background (not shown).

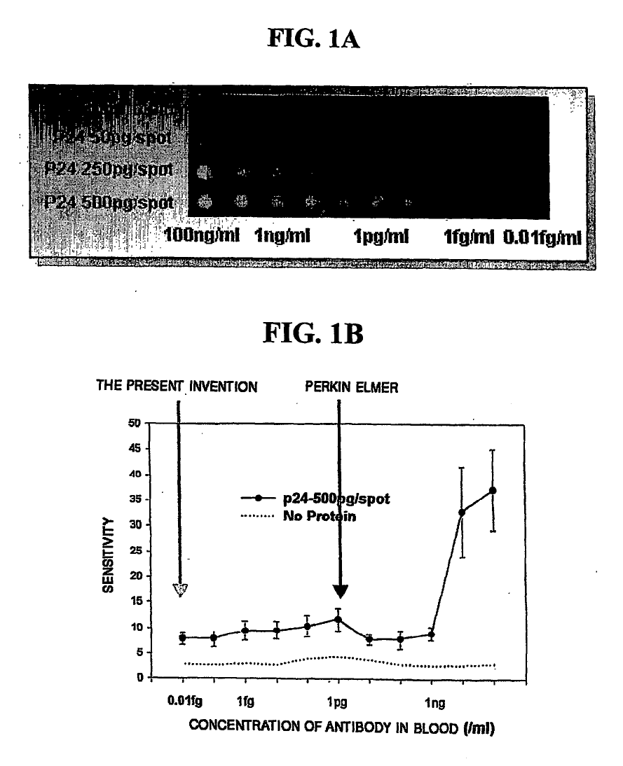

A biochip was prepared by adding HIV P24 protein practically used in the AIDS diagnosis to the sol mixture solution according to Example 2 (Table 1, Composition 5) and was examined for the reaction with antibody in the blood of an AIDS patient. FIG. 1a shows the results of the experiment, in which P24 protein immobilized on the biochip has reacted with the HIV antibody in the blood and the signals were recognized by the Cy3-labeled antibody. When the sol mixture solution contained no protein, as a control, no reaction occurred.

On the basis of the above results, AIDS antigen with a known concentration was sequentially diluted from 100 ng/ml to determine the limit of detection at which the antigen in the blood can be measured. With the chip prepared according to the present invention, it was possible to observe a signal 5 times or more as compared to the background, down to a concentration of 0.01 fg/ml. According to the graph shown in FIG. 1b, the biochip of the present invention had 10,000 times improved sensitivity as compared to Hydrogel chip (1 pg/ml) of PerkinElmer.

Experiment 3: Examination of Spot Formed by Gelation on Protein Chip

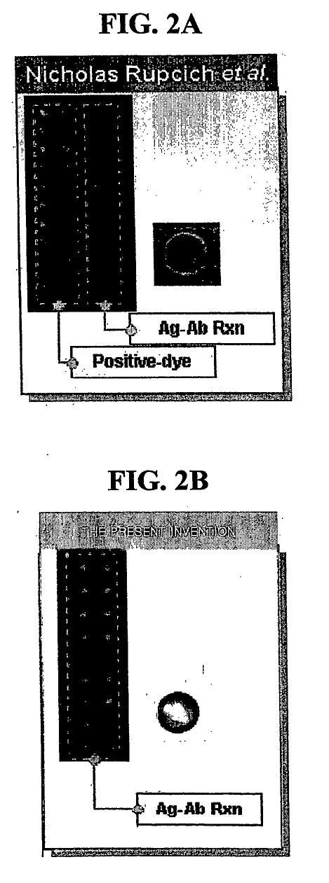

In order to examine the protein spots integrated on the protein chip surface for transparency, cracks and morphology, the integrated spots were observed under an optical microscope and CLSM, and the results are shown in FIG. 2b.

The spots were transparent and had no crack. Upon an image analysis after the antigen-antibody reaction, it was observed that the spots had a uniform morphology. As shown in FIG. 2a, the morphology and transparency of the spots according to the present invention attained superiority over other technologies (Chem. Mater., 15 (9), 1803 -1811, 2003).

Experiment 4: Confirmation of Stability of Protein Chip Prepared by Gelation on Chip

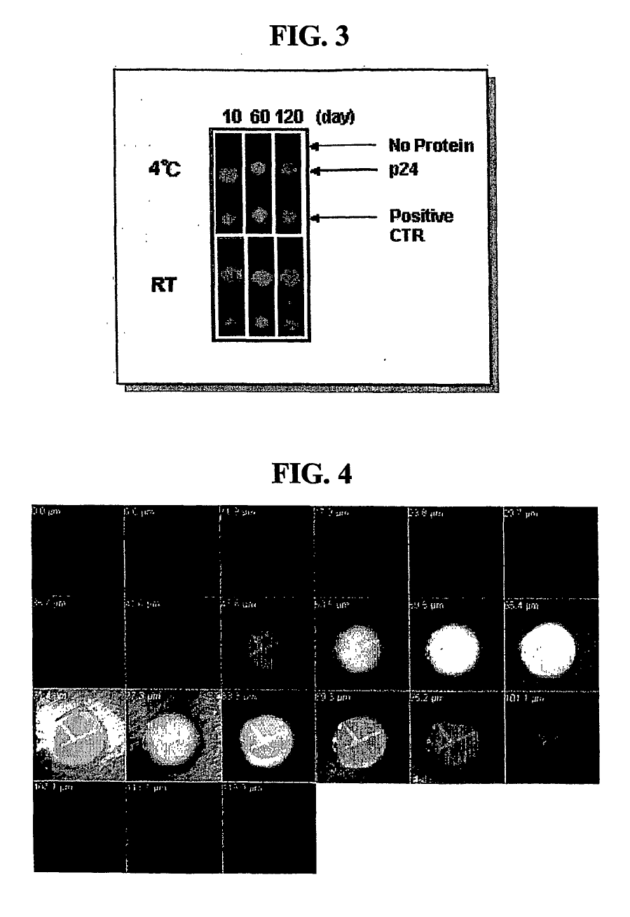

As shown in FIG. 3, for a period up to 4 months, when the same spots was subjected to the antigen-antibody reaction, the sensitivity was uniformly maintained in the range of about 5% of sensitivity change, without regard to 4° C. or 25° C. Also, the spots formed by the gelation according to the present invention were stable for more than 6 months (not shown) and thus it was confirmed that the present invention can be manufactured into products.

Experiment 5: Distribution of Reactive Proteins in Spots by Gelation on Chip

This experiment was conducted to confirm that the proteins 3-dimensionally supported on the surface by the gelation on the chip were evenly distributed in a spot. The chip prepared in Experiment 2 was examined for protein distribution using CLSM to confirm the 3-dimensional structure of the spots. The results of the experiment are shown in FIG. 4. It was confirmed that in the spots having a thickness of about 100 to 300 um, the fluorescent-labeled proteins were not attached to the outer surface or the bottom but evenly distributed inside the spots.

### EXAMPLE 5

### Preparation of Protein Chip for Diagnosis and Antigen-Antibody Reaction for Diagnosis

Experiment 1: Protein Chip Comprising Antigen for HIV Diagnosis

Following the same procedures as the method used in Example 2, the protein-sol mixture (Table 1, Composition 5) was gelled on the chip, wherein the used proteins were purified HIV p24 protein (1 μg/μl), combo protein (1 μg/μl) comprising p24 used for HIV diagnosis, HIV polymerase RgpIII (1 μg/μl) and BSA (1 μg/μl).

To obtain quantitative results, each protein was sequentially diluted by 10 times and the most suitable concentration condition for integration (40 pg-4 ng/spot) was determined.

The conditions and procedures of the AIDS diagnosis reaction to sense HIV antigen in the human serum using P24 protein, an indicating factor for HIV diagnosis, are as follows. In order to detect HIV p24, anti-p24 as a primary antibody was reacted at 25° C. for 30 minutes and then washed. Cy3-conjugated anti-rabbit IgG (Sigma-Aldrich Company) as a secondary antibody was reacted for 30 minutes under the same incubation conditions used for the reaction with the primary antibody, washed and completely dried in the air. The Cy3 signal was detected using a scanner (Exon).

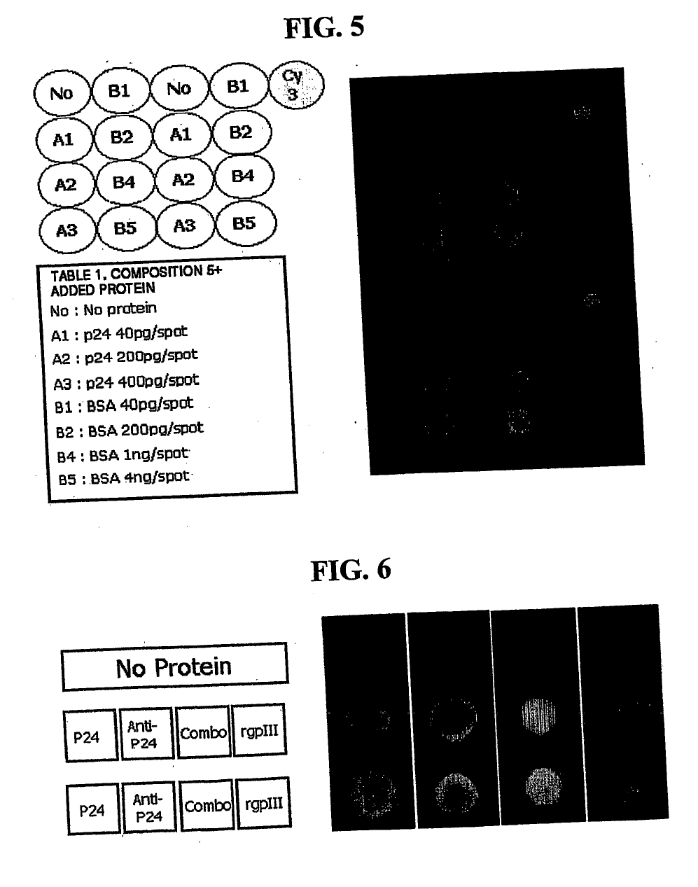

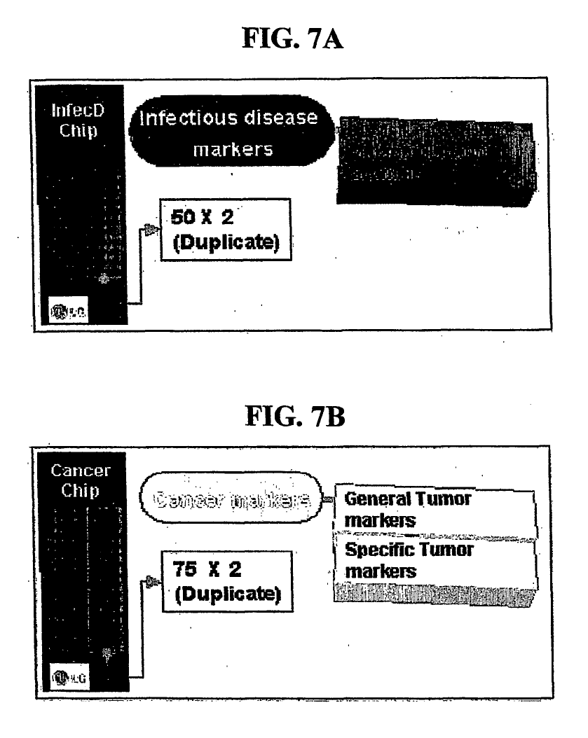

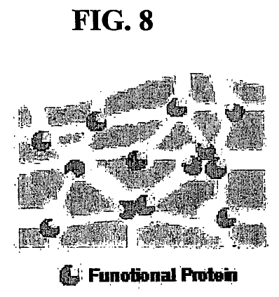

As a result, spots without a protein or spots with BSA protein (which is not related to HIV) did not show a signal, while spots containing P24 showed concentration-dependent signals. Even at a concentration of about 40 pg, the detection can be suitably conducted (FIG. 5).

Also, combo protein containing P24, HIV polymerase and RgpIII showed signals as indicated in FIG. 6.

From the above results, it was noted that the antigen-antibody reaction could specifically occur on the protein chip prepared according to the present invention.

Experiment 2: Immobilization of Antibody

In Experiment 1, only antigen proteins were immobilized on the protein chip. However, it was observed that when a sol mixture containing an antibody by adjusting the sol composition was used, the immobilized antibody could undergo an antigen-antibody reaction. Here, the used antibody was monoclonal anti-P24 antibody used for AIDS diagnosis. The protein chip with the monoclonal anti-P24 antibody immobilized was reacted with a blood AIDS protein and subjected to the Sandwich detection including detections with primary and secondary antibodies.

FIG. 6 shows the result of the experiment, in which the biochips were prepared by adding each of the indicating proteins to the Composition 5 in Table 1, for antigen and the antibodies to the Composition 7 in Table 1, for antibody, and subjected to the AIDS diagnosis as in Experiment 1.

In all duplicate spots of the indicating antigen P24 for HIV diagnosis, combo and rgpIII respectively, the antigens were recognized by the HIV antibody while in the spot without containing a protein, no signal was observed. Also, in case of anti-P24 using the antibody as a diagnosis indicator, the antibody was detected by the HIV antigen.

In the spot without containing an antibody, no signal was observed. FIG. 6 shows that the biochip according to the present invention does not diagnose alternatively either antigen or antibody but can diagnose both antigen and antibody on the same chip under the same condition, which makes the biochip of the present invention distinguishable from the conventional diagnosis chips.

So far, the present invention has been described of the preferred embodiment, however various modification can be made without departing the scope of the present invention. Therefore, the scope of the present invention is not limited to the above described embodiments but defined by the scope of the claims and equivalence thereof

1. A biochip wherein a gel type of spots are integrated and immobilized on a chip substrate with biomaterials entrapped in pores therein and encapsulated by spot.

2. The biochip according to claim 1, which is used as protein chips, DNA chip, new drug screening chips, environmental assay chips, toxicity assay chips, or food bacteria assay chips.

3. A coating solution for a chip substrate comprising a coating agent selected from the group consisting of polyvinyl acetate (PVAc) having a molecular weight in the range of 800 to 200,000, poly(vinyl butyral-co-vinylalcohol-co-vinyl acetate) having a molecular weight in the range of 70,000 to 120,000, poly(methyl methacrylate-co-methacrylic acid) having a molecular weight of 10,000 or more, poly(methyl vinyl ether-maleic anhydride) having a molecular weight of 200,000 or more, poly(methyl vinyl ether-maleic anhydride) having a molecular weight of 1,000,000 or more, poly(methyl acrylate) having a molecular weight of 10,000 or more, 3-glycidoxypropyltrimethoxysilane (GPTMOS), dissolved in solvent(s) selected from the group consisting of methylene chloide, tetrahydrofuran, ethanol, methanol, butanol, methyl ethyl ketone, acetone, isopropyl alcohol, ethyl acetate, methyl isobutyl ketone, and di-acetone alcohol.

4. The coating solution according to claim 3, wherein the solvent is used in a concentration of 5 to 20% by weight of the total coating solution.

5. A chip substrate coated with the coating solution according to claim 3.

6. The chip substrate according to claim 5, wherein the coating is performed by spin coating.

7. The chip substrate according to claim 5, which is selected from the group consisting of polymethyl methacrylic acid, polycarbonate and cyclic olefin copolymers.

8. The chip substrate according to claim 5, which has a slide shape.

9. A method for preparing a biochip comprising (1) integrating a sol mixture containing biomaterials in the shape of spots on a surface treated chip substrate; and (2) gelling the sol mixture in the shape of spots on the chip substrate.

10. The method according to claim 9, wherein the chip substrate as defined in claim 5 is used.

11. The method according to claim 10, wherein the sol mixture comprises at least one selected from the group consisting of silicate monomers, poly glyceryl silicate (PGS), 3-glycidoxypropyltrimethoxysilane (GPTMOS) and (N-triethoxysilylpropyl)-O-polyethylene oxide urethane (PEOU), as a basic component for the sol-gel matrix.

12. The method according to claim 11, wherein the silicate monomer is at least one selected from the group consisting of tetramethyl orthosilicate (TMOS), tetraethyl orthosilicate (TEOS), methyltrimethoxysillane (MTMS), ethyltriethoxysilane (ETEOS), trimethoxysilane (TMS), and 3-aminopropyltrimethoxysilicate (APTMOS).

13. The method according to claim 11, wherein the sol mixture farther comprises at least one selected from the group consisting of glycerol, polyethylene glycol having a molecular weight of 400 to 8000, as the basic component for the sol-gel matrix.

14. The method according to claim 11 or 13, wherein the basic component for the sol-gel matrix is used in the range of 30 to 60% by volume of the total sol mixture.

15. The method according to claim 11, wherein the silicate monomer is used in the range of 10 to 40% by volume of the total sol mixture.

16. The method according to claim 11 or 13, wherein poly glyceryl silicate (PGS), 3-glycidoxypropyltrimethoxysilane (GPTMOS), (N-triethoxysilylpropyl)-O-polyethylene oxide urethane (PEOU), glycerol and polyethylene glycol (PEG) are used in the range of 2 to 10% by volume of the total sol mixture.

17. The method according to claim 16, wherein PGS is used in the range of 0.5 to 6% by volume, GPTMOS is used in the range of 1 to 10% by volume for, PEOU is used in the range of 5 to 15% by volume; glycerol is used in the range of 1 to 5% by volume, or PEG is used in the range of 1 to 6% by volume, based on the total sol mixture.

18. The method according to claim 11, wherein the polyglyceryl silicate (PGS) is a polymerization intermediate from the reaction of silicate monomer and glycerol.

19. The method according to claim 11, wherein the sol mixture further comprises a pH buffer.

20. The method according to claim 19, wherein the pH buffer is phosphate buffer.

21. The method according to claim 19, wherein the pH buffer has a pH range of 4 to 9.

22. The method according to claim 19, wherein the concentration of the pH buffer is in the range of 5 to 100 mM.

23. The method according to claim 9, wherein the conditions for the gelation includes a temperature of 4° C. to 25 ° C. and a humidity of 40 to 80%.

24. A method for assaying a binding between a biomaterial immobilized on a biochip and a target material, comprising the steps of
applying a sample containing the target material to be assayed for binding with the biomaterial to the biochip as defined in claim 1 or the biochip prepared by the method as defined in claim 9; and
detecting the target material specifically bound to the bio material.

25. The method according to claim 24, wherein the reaction between the biomaterial and the target material occurs in the pores in the gel type spots wherein the biomaterial are entrapped in the pores and encapsulated by spot.
